(19) United States
(12) Patent Application Publication
(10) Pub. No.: US 2023/0017547 A1
(43) Pub. Date: Jan. 19, 2023

(54) DIGITAL MICROFLUIDIC (DMF) SYSTEM, DMF CARTRIDGE, AND METHOD INCLUDING INTEGRATED OPTICAL FIBER SENSING

(71) Applicant: Nicoya Lifesciences, Inc., Kitchener (CA)

(72) Inventors: Krishna IYER, Waterloo (CA); Gordon H. HALL, Milton (CA); Champika SAMARASEKERA, Guelph (CA); Ryan Cameron DENOMME, Kitchener (CA)

(21) Appl. No.: 17/814,208

(22) Filed: Jul. 21, 2022

Related U.S. Application Data
(63) Continuation of application No. PCT/CA2021/050059, filed on Jan. 20, 2021.
(60) Provisional application No. 62/964,424, filed on Jan. 22, 2020.

(51) Int. Cl. B01L 3/00; G01N 21/64; G01N 33/543

(52) U.S. Cl. CPC B01L 3/502792; G01N 21/6428; G01N 33/54386; G01N 2021/6432; G01N 2201/08; B01L 2300/0654; B01L 2300/0645; B01L 2400/0427; B01L 2200/04; B01L 2200/0663

(57) ABSTRACT

A cartridge for use with an instrument to perform measurement of a fluid, including a digital microfluidics substrate comprising a plurality of electrowetting electrodes operative to perform droplet operations on a liquid droplet in a droplet operations gap; a top plate separated from the digital microfluidics substrate to form a droplet operations gap and comprising openings for injecting liquids into the droplet operations gap; a fiber assembly comprising a fiber optic probe projecting into the droplet operations gap and having a sensing end situated in proximity with one or more of the electrowetting electrodes.

### CROSS-REFERENCE TO RELATED APPLICATIONS

This patent application is a continuation PCT/CA2021/050059 filed Jan. 20, 2021, which claims priority to U.S. Patent App. No. 62/964,424, filed on Jan. 22, 2020, which are incorporated herein by reference.

### TECHNICAL FIELD

The present disclosure relates to a digital microfluidic (DMF) system, DMF cartridge, and method making use of integrated optical fiber sensing.

### BACKGROUND

Digital microfluidics systems are useful for manipulating droplets for a variety of lab-on-a-chip applications. Optical sensing techniques are commonly used with digital microfluidics systems, e.g., for detection of analytes in droplets. Simple fiber optic sensors have been described for use in detecting light in digital microfluidics systems. However, there is a need in the art to enhance the capabilities of fiber optic sensing for conducting complex analyses using digital microfluidics.

### SUMMARY

The present disclosure provides a cartridge for use with an instrument to perform measurement of a fluid, an instrument for operating the cartridge, and methods of making and using the cartridge. The present disclosure also includes a fiber assembly which is useful for assembling a cartridge of the present disclosure.

In one embodiment, the cartridge includes digital microfluidics comprising a plurality of electrowetting electrodes operative to perform droplet operations on a liquid droplet in a droplet operations gap. The cartridge may also include a fiber optic probe projecting into the droplet operations gap into proximity with a set of two or more of the electrowetting electrodes such that a droplet situated atop any electrode of the set of two or more electrodes will contact the fiber optic probe.

In another embodiment, the cartridge includes digital microfluidics substrate comprising a plurality of electrowetting electrodes operative to perform droplet operations on a liquid droplet in a droplet operations gap and a top plate separated from the digital microfluidics substrate to form a droplet operations gap and comprising openings for injecting liquids into the droplet operations gap. The cartridge may also include a fiber assembly comprising a fiber optic probe projecting into the droplet operations gap and having a sensing end situated in proximity with one or more of the electrowetting electrodes.

In certain embodiments, a fiber optic probe projecting into the droplet operations gap is situated in proximity with a set of two or more of the electrowetting electrodes such that a droplet situated atop any electrode of the set of two or more electrodes will contact the fiber optic probe. The probe may include a ligand. A droplet in the cartridge is controllable by the electrowetting electrodes to contact the fiber optic probe. A low viscosity oil or other filler substance (e.g., filler fluid) may be used to fill the gap around the droplet.

The present disclosure also provides a method of conducting an assay. The method may include providing a fiber optic probe comprising a ligand; providing a droplet having a volume which is less than about 1000 nL and comprising an analyte potentially having an affinity for the ligand; contacting the droplet with the end of the probe and oscillating the droplet without removing the droplet from contact with the probe. In some cases, the oscillation ranges from about 0.5 to about 15 Hz, or from about 4 to about 10 Hz. In some cases, the droplet has a volume which is less than about 900 nL, or less than about 800 nL, or less than about 700 nL, or less than about 600 nL, or less than about 500 nL, or less than about 400 nL. In some cases, the droplet has a volume between 200 and 400 nL.

In some cases, the fiber optic probe comprises multiple ligands and the droplet comprises multiple analytes. In some cases, the method comprises providing multiple fiber optic probe and multiple droplets and contacting each of the multiple droplets with a corresponding fiber optic probe and oscillating each of the multiple droplets in contact with the corresponding fiber optic probe. In another embodiment, a single droplet can be transported from one probe to another for a series of assays.

In some cases, the oscillation is mediated by electrowetting electrodes. In some cases, the oscillation is mediated by electrowetting electrodes in a droplet operations gap of a droplet actuator or electrowetting cartridge. In some cases, the fiber optic probe is aligned so that the end is adjacent to a droplet operations electrode. In some cases, the fiber optic probe is aligned so that the end is in proximity to an edge of a droplet operations electrode. In some cases, the oscillation is substantially at a right angle to a line extending along the length of the fiber optic probe. In some cases, the oscillation is substantially in line with a line extending along the length of the fiber optic probe. In some cases, the oscillation is multidirectional. In some cases, the oscillation is multidirectional in a plane parallel to a line extending along the length of the fiber optic probe. In some cases, the oscillation is conducted using an elongated droplet, e.g., 2X, 3X, 4X or longer, where X is the number of electrowetting electrodes used to elongate the droplet.

In some cases, the assay is selected from the following: molecular library screening assays, binding kinetics assays, affinity determination assays, binding site mapping assays, competition analysis assays, specificity determination assays, and characterizations of antibody binding, and combinations of the foregoing. In some cases the assay produces a response curve.

### BRIEF DESCRIPTION OF DRAWINGS

Having thus described the subject matter in general terms, reference will now be made to the accompanying drawings, which are not necessarily drawn to scale, and wherein:

FIG. 1 illustrates a block diagram of an example of the DMF system that includes integrated optical fiber sensing;

FIG. 2A and FIG. 2B illustrate an example of a DMF cartridge in relation to a DMF instrument and showing an example of an optical fiber interface;

FIG. 3, FIG. 4, FIG. 5 are side views of an example of a portion of the DMF cartridge of the DMF system and wherein a fiber optic probe is introduced into the gap from the top, bottom, and side, respectively;

FIG. 6 is a side view of an example of a portion of the DMF cartridge of the DMF system and wherein a fiber optic probe is introduced into the gap and optical sensing operations are performed in reflection mode;

FIG. 7A and FIG. 7B illustrate side views of an example of a portion of the DMF cartridge of the DMF system and wherein a sensor surface is provided on the tip of the fiber optic probe;

FIG. 8 illustrates a side view of an example of a portion of the DMF cartridge of the DMF system and wherein one or more optical elements is provided on the tip of the fiber optic probe;

FIG. 9 illustrates a side view of an example of the DMF cartridge of the DMF system that includes both a primary and secondary optical measurement device;

FIG. 10 illustrates a flow diagram of an example of a method of using the DMF system and/or DMF cartridge that includes integrated optical fiber sensing;

FIG. 11 illustrates an example of a sample droplet oscillating back and forth atop the droplet operations electrodes and in contact with the fiber optic probe;

FIG. 12A and FIG. 12B illustrate a top perspective view and a bottom perspective view, respectively, of an example instantiation of the DMF cartridge that includes integrated fiber sensing;

FIG. 13A and FIG. 13B illustrate a top exploded view and a bottom exploded view, respectively, of an example instantiation of the DMF cartridge that includes integrated fiber sensing;

FIG. 14A and FIG. 14B illustrate various views of an example of a fiber assembly of the DMF cartridge;

FIG. 15 shows an example of a process of fiber sensing prototyping;

FIG. 16 shows an electron micrograph of an example of a nanoparticle sensor surface; and

FIG. 17A and FIG. 17B show plots of example test results of determining the affinity of protein-A and IgG using an optical fiber-based surface plasmon resonance detection method within a DMF device.

### DETAILED DESCRIPTION

The invention relates to digital microfluidic (DMF) systems, DMF cartridges, and methods including integrated optical fiber sensing.

A DMF system and DMF cartridge may include, for example, a fiber optic probe directly inserted into the droplet operations gap of a DMF cartridge. The DMF system may include, for example, the DMF cartridge, one or more illumination sources, one or more optical measurement devices, and a controller. The DMF cartridge may further include a fiber optic probe directly inserted into the droplet operations gap of the DMF cartridge.

In some embodiments, the DMF system, DMF cartridge, and method provide a fiber optic probe wherein the tip of the fiber optic probe is directly inserted into the droplet operations gap of a DMF cartridge.

In some embodiments, the DMF system, DMF cartridge, and method may provide a fiber optic probe inserted through the top, bottom, and/or side of a DMF cartridge.

In some embodiments, the present disclosure provides a single fiber optic probe that may act as the conduit for light both into and out of a sensing region in the droplet operations gap of a DMF cartridge.

The droplets used in assays of the present disclosure may be much smaller than in many traditional assays, e.g., less than about 1000 nL, or less than about 900 nL, or less than about 800 nL, or less than about 700 nL, or less than about 600 nL, or less than about 500 nL, or less than about 400 nL, or less than about 300 nL, or less than about 200 nL, or less than about 100 nL.

The droplets used in SPR assays of the present disclosure may be much smaller than in many traditional SPR assays, e.g., less than about 1000 nL, or less than about 900 nL, or less than about 800 nL, or less than about 700 nL, or less than about 600 nL, or less than about 500 nL, or less than about 400 nL, or less than about 300 nL, or less than about 200 nL, or less than about 100 nL.

The droplets used in SPR biomolecular interaction assays of the present disclosure may be much smaller than in many traditional SPR biomolecular interaction assays, e.g., less than about 1000 nL, or less than about 900 nL, or less than about 800 nL, or less than about 700 nL, or less than about 600 nL, or less than about 500 nL, or less than about 400 nL, or less than about 300 nL, or less than about 200 nL, or less than about 100 nL. Examples of SPR biomolecular interaction assays include molecular library screening assays, binding kinetics assays, affinity determination assays, binding site mapping assays, competition analysis assays, specificity determination assays, and characterizations of antibody binding.

In some embodiments, the present disclosure may provide one or more illumination sources for providing excitation light into the fiber optic probe and one or more optical measurement devices for receiving and processing emission light from the same fiber optic probe.

In some embodiments, the present disclosure may provide one or more illumination sources for providing excitation light using free-space optics and one or more fiber optic probes for receiving and processing emission light. Similarly, in some embodiments the present disclosure may provide one or more illumination sources for providing excitation light coupled into the fiber optic probe and one or more optical measurement devices for receiving and processing the emission light using free-space optics.

In some embodiments, the present disclosure provides a fiber optic probe directly inserted into the droplet operations gap of a DMF cartridge and wherein optical sensing operations may occur in reflection mode, transmission mode, or both reflection mode and transmission mode.

In some embodiments, the present disclosure provides other components and/or elements (e.g., sensing layers, optical elements) on the tip of a fiber optic probe in the droplet operations gap of a DMF cartridge.

Additionally, the present disclosure provides methods of using the DMF system and DMF cartridge where integrated optical fiber sensing is provided.

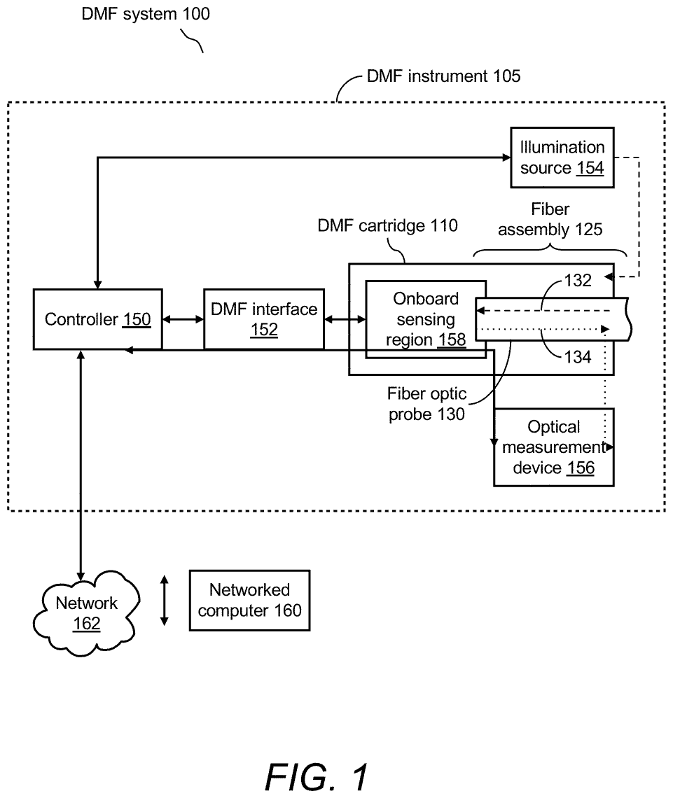

FIG. 1 is a block diagram of an example of the DMF system 100 that includes integrated optical fiber sensing. DMF system 100 may be, for example, a plasmon resonance (PR) system and/or a localized surface plasmon resonance (LSPR) system for analysis of analytes. Analysis can mean, for example, detection, identification, quantification, or measuring analytes and/or the interactions of analytes with other substances, such as binding kinetics and/or thermodynamics. Exemplary analytes may include, but are not limited to, small molecules, proteins, peptides, atoms, and ions. For example, DMF system 100 may be used to measure the binding kinetics of a ligand to a macromolecule, such as a receptor.

DMF system 100 may similarly be configured as a reflective interferometric sensor such as a bio-layer interferometry (BLI) or single color reflectometry (SCORE). These interferometric sensors are deposited using techniques such as chemical vapor deposition (CVD) or physical vapor deposition (PVD) onto the tip of an optical fiber with typical materials used for thin film coatings such as Zinc Sulfide, Titanium Dioxide, Magnesium Fluoride, Silicon Dioxide with an ultimate metallic layer of plasmonic metals such as gold. For example, a ligand may be immobilized on the tip of the fiber. The presence or absence of an analyte interacting with the ligand will cause a wavelength shift or change in interference pattern with respect to light reflected from the sensor. Analysis can mean, for example, detection, identification, quantification, or measuring analytes and/or the interactions of analytes with other substances, such as binding kinetics. Exemplary analytes may include, but are not limited to, small molecules, proteins, peptides, atoms, and ions. For example, DMF system 100 may be used to measure the binding kinetics of a ligand to a macromolecule, such as a receptor. In one analytical embodiment, light reflected by a BLI fiber sensor exposed to the analyte can be compared with light reflected by a BLI fiber sensor not exposed to the analyte.

DMF system 100 may similarly be configured as a fluorescent measurement system. The sensor in this case may be an unmodified sensor or the fluorophore could be immobilized onto the tip of the fiber. In another example, the tip of the fiber may be coated with coating that modifies the emissive properties of the fluorophore by quenching or by energy transfer such as Forster Resonant Energy Transfer. One or multiple fluorophores may be used in conjunction with each other simultaneously. Such a setup could measure the intensity and/or the lifetime of fluorescence. The sample exhibiting fluorescence could either be in the solution or captured at the tip of the optical fiber. The fluorescence could be intrinsic to the sample or generated using fluorophores such as small molecules, quantum dots etc. that bind to or otherwise interact with the analyte. Analysis can mean, for example, detection, identification, quantification, or measuring analytes and/or the interactions of analytes with other substances, such as binding kinetics. Exemplary analytes may include, but are not limited to, small molecules, proteins, peptides, atoms, and ions. For example, DMF system 100 may be used to measure the binding kinetics of a ligand to a macromolecule, such as a receptor.

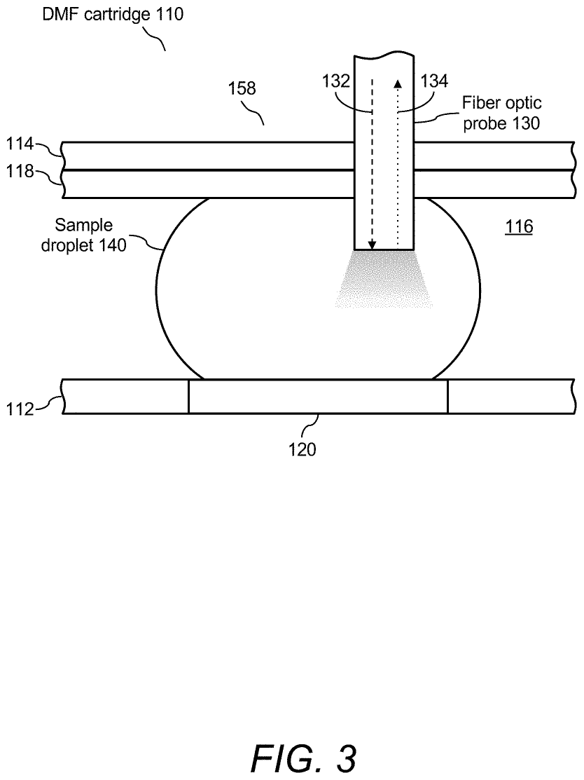

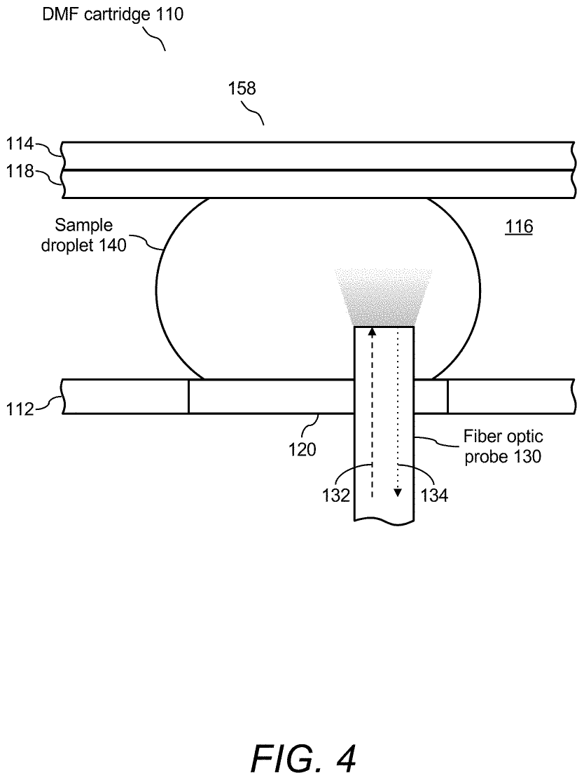

DMF system 100 features the integration of optical fiber-based sensors with digital microfluidics. For example, DMF system 100 may include a DMF cartridge 110. DMF cartridge 110 may be, for example, a droplet actuator device that provides DMF capabilities generally for conducting droplet operations, such as merging, splitting, dispensing, and diluting droplets. One application of these DMF capabilities is sample preparation. However, the DMF capabilities may be used for other processes, such as waste removal or flushing between runs. DMF cartridge 110 may include an onboard sensing region 158 that may be coupled to a fiber optic probe 130. In DMF cartridge 110, a single fiber optic probe 130 may act as the conduit for light both into and out of onboard sensing region 158. Fiber optic probe 130 may include one optical fiber or a bundle of multiple optical fibers. More details of DMF cartridge 110, onboard sensing region 158, and fiber optic probe 130 are shown and described below with reference to FIG. 3 through FIG. 9.

In various embodiments, the sensing end of fiber optic probe 130 is aligned so that the end is adjacent to droplet operations electrode 120. In various embodiments, the end of fiber optic probe 130 is aligned so that the end is in proximity to an edge of droplet operations electrode 120. In various embodiments, the end of fiber optic probe 130 is aligned so that the end is aligned with an edge of droplet operations electrode 120.

DMF system 100 may further include a controller 150, a DMF interface 152, an illumination source 154, and an optical measurement device 156. Controller 150 may be electrically coupled to the various hardware components of DMF system 100, such as to DMF cartridge 110, illumination source 154, and optical measurement device 156. For example, controller 150 may be electrically coupled to DMF cartridge 110 via DMF interface 152, wherein DMF interface 152 may be, for example, a pluggable interface for connecting mechanically and electrically to DMF cartridge 110. Together, DMF cartridge 110, controller 150, DMF interface 152, illumination source 154, and optical measurement device 156 form a DMF instrument 105.

Controller 150 may, for example, be a general-purpose computer, special purpose computer, personal computer, microprocessor, or other programmable data processing apparatus. Controller 150 serves to provide processing capabilities, such as storing, interpreting, and/or executing software instructions, as well as controlling the overall operations of DMF system 100. Controller 150 may be configured and programmed to control data and/or power aspects of these devices. For example, controller 150 controls droplet manipulation in DMF cartridge 110 by activating/deactivating electrodes. Generally, controller 150 may be used for any functions of DMF system 100. For example, controller 150 may be used to authenticate the DMF cartridge 110 in a fashion similar to how printer manufacturers check for their branded ink cartridges, controller 150 may be used to verify that the DMF cartridge 110 is not expired, controller 150 may be used to confirm the integrity of the DMF cartridge 110 by running a certain protocol for that purpose, and so on.

Controller 150 may include one or more input interfaces that connect processing units to input devices. Input interfaces allow users of DMF system 100 to communicate commands to processor(s). One such exemplary command is the execution of program code. Input devices may take the form of keyboards, mouse devices, voice-activated systems, touch screens, and/or other suitable devices as will be known to a person of skill in the art.

In some embodiments, controller 150 may include one or more output interfaces connecting processing units to output devices, such as a graphical user interface (GUI). This enables DMF system 100 to communicate results of various processing operations to users, such as experiment results. Software instructions may be stored in memory unit(s) of controller 150 and may include conventional semiconductor random access memory (RAM) or other forms of memory known in the art; and/or software instructions may be stored in the form of program code on one or more computer readable-storage media, such as a hard drive, USB drive, read/write CD-ROM, DVD, tape drive, flash drive, optical drive, etc. These instructions may be executed in response to a user's interaction with DMF system 100 via an input device.

In some embodiments, DMF cartridge 110 may include capacitive feedback sensing. Namely, a signal coming from a capacitive sensor that can detect droplet position and volume. Further, in other embodiments, instead of or in addition to capacitive feedback sensing, DMF cartridge 110 may include a camera to provide optical measurement of the droplet position and volume, which can trigger controller 150 to re-route the droplets at appropriate positions.

In some embodiments, DMF cartridge 110 may include heating zones (not shown). It will be appreciated that a variety of sample prep steps and assays will benefit from temperature control. As described in U.S. Pat. No. 8,658,111, incorporated herein by reference, thermal control may generally be provided in three ways: (1) thermal control of the entire DMF cartridge 110; (2) thermal control of a region of a DMF cartridge 110 using a heater that is in contact with or in proximity to the controlled region; and (3) thermal control of a region of the DMF cartridge 110 or the entire DMF cartridge 110 using a heater that is integrated into the DMF cartridge 110 (e.g., in the substrate comprising the path or array of electrodes and/or in a top substrate of the DMF cartridge 110, when present). Combinations of the foregoing approaches are also possible. Examples of heating techniques include heater bars mounted on the instrument adjacent to the cartridge, and heaters integrated into the cartridge itself.

In an integrated heater approach, temperature zones can be created and controlled using thermal control elements directly integrated into the DMF cartridge 110. Thermal control elements (heating and/or cooling) may be integrated on the bottom substrate and/or top substrate (when present) of the DMF cartridge 110 and on the bottom and/or top surface of either substrate, or integrated within the structure of either substrate, or arranged between substrates. In some cases, thermal control elements may be electronically coupled to and controlled by controller 150. Thermal zones may be created using distinct heating elements and may thus serve as a distinct thermal zones within the DMF cartridge 110. This arrangement permits multiple steps in an analysis, such as sample preparation and thermal cycling, requiring different temperatures to be performed simultaneously at different temperatures in different thermal zones on a DMF cartridge 110. For example, using electrowetting-mediated droplet operations droplets can be physically transported or shuttled between thermal zones of different fixed temperatures to perform thermal cycling for an amplification reaction.

In one embodiment, heaters in the thermal zones may be formed using thin conductive films. Examples of suitable thin films include Pt heater wires and transparent indium-tin-oxide (ITO). In one embodiment, metal (e.g., copper) vias in a substrate of the DMF cartridge 110 are used.

An thermocouple for temperature regulation can also be used to control temperature in a thermal zone. A temperature probe may be used to measure the temperature in the thermal zone and provide temperature measurements to the controller so that the controller can precisely control the temperature of the relevant thermal zone.

DMF instrument 105 may be connected to a network. For example, controller 150 may be in communication with a networked computer 160 via a network 162. Networked computer 160 may be, for example, any centralized server or cloud server. Network 162 may be, for example, a local area network (LAN) or wide area network (WAN) for connecting to the internet.

In DMF system 100, illumination source 154 and optical measurement device 156 may be arranged with respect to onboard sensing region 158 and/or fiber optic probe 130 of DMF cartridge 110. Illumination source 154 may be, for example, a light source for the visible range (wavelengths 400-800 nm), such as, but not limited to, a white light-emitting diode (LED), a halogen bulb, an arc lamp, an incandescent lamp, and lasers. Illumination source 154 is not limited to a white light source. Illumination source 154 may be any color light that is useful in DMF system 100. Illumination source 154 supplies excitation light 132 to onboard sensing region 158 of DMF cartridge 110.

Optical measurement device 156 may be any optical transducer device used to obtain, for example, light intensity readings. Optical measurement device 156 may be, for example, a charge coupled device, a photodetector, a spectrometer, a photodiode array, a camera, or any combinations thereof. Further, DMF system 100 is not limited to one illumination source 154 and one optical measurement device 156 only. DMF system 100 may include multiple illumination sources 154 and/or multiple optical measurement devices 156 in order to support any detection operations in DMF system 100 and/or DMF cartridge 110. Optical measurement device 156 receives and processes emission light 134 from onboard sensing region 158 of DMF cartridge 110. Accordingly, a single fiber optic probe 130 of DMF cartridge 110 may act as the conduit for both excitation light 132 into onboard sensing region 158 and emission light 134 out of onboard sensing region 158.

Components of DMF system 100 and/or DMF instrument 105 may be optically coupled to onboard sensing region 158 and/or fiber optic probe 130 of DMF cartridge 110 and may also be decoupled. The optical coupling/decoupling may be, for example, fiber optic connectors, fiber optic couplers, and/or free-space optical couplers.

Conventional methods of measurement of optical stimuli from a DMF device use free-space optics to capture stimuli. The limitations of free-space optics in this context are primarily owing to relatively poorer performance as a result of stray light and light losses. The poor performance is often overcome with the use of optical components such as lenses, filters and other similar optical components, however these components drive up the cost of the system. Compared with conventional methods, the integration of optical fiber sensors with DMF provides certain benefits. For example, the benefits of DMF system 100 that features the integration of optical fiber-based sensors with digital microfluidics may include, but are not limited to, allows for localized optical interrogation techniques, provides a low-cost method of achieving high optical performance (e.g., reduced component count for lower cost), limits light loss in the system for improved signal, and limits collection of stray light for lower noise.

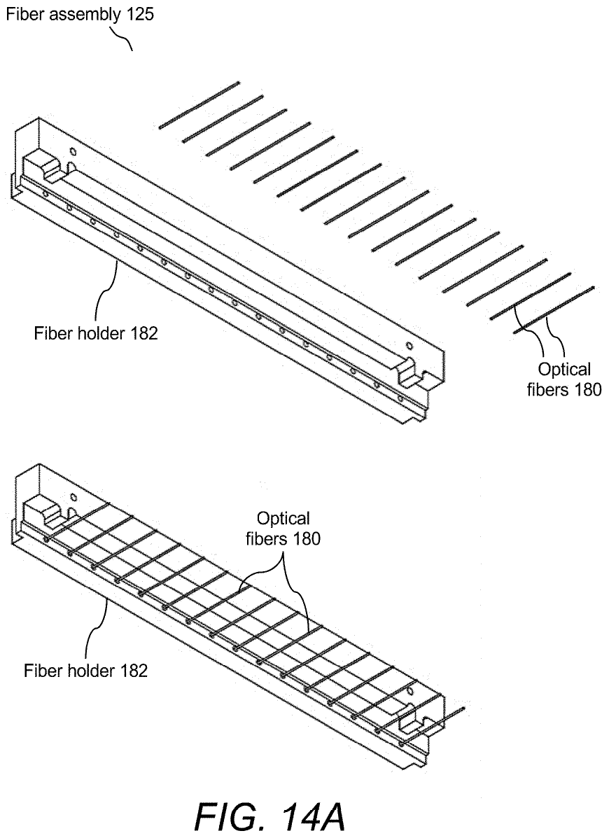

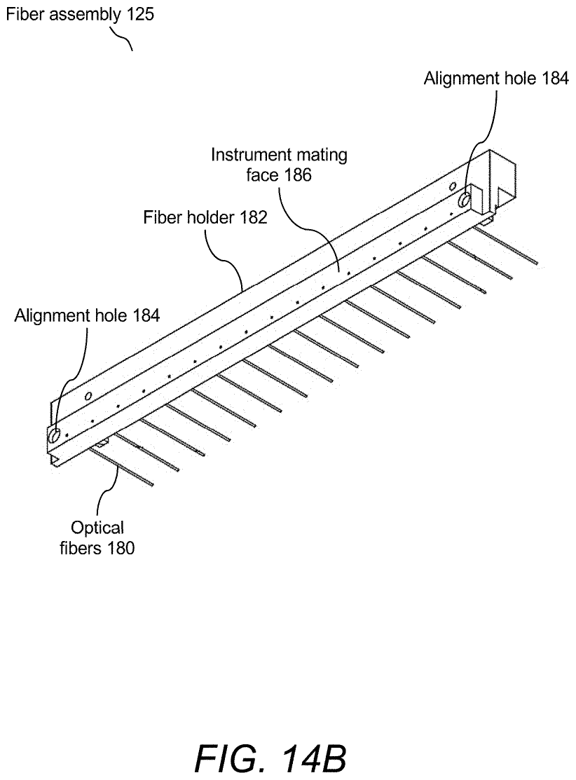

In DMF system 100, fiber optic probe 130 is integrated into DMF cartridge 110 such that the tip of fiber optic probe 130 is present directly in the droplet operations gap of DMF cartridge 110 (see FIG. 3 through FIG. 9). In this way, the tip of fiber optic probe 130 may interact directly with the droplets being processed in DMF cartridge 110. Accordingly, the tip of fiber optic probe 130 forms onboard sensing region 158, wherein DMF cartridge 110 is able to move droplets to and from onboard sensing region 158. DMF system 100 and/or DMF cartridge 110 is not limited to one fiber optic probe 130 and one onboard sensing region 158 only. This is exemplary only. DMF system 100 and/or DMF cartridge 110 may include any number of fiber optic probes 130 and/or onboard sensing regions 158. Further, the one or more fiber optic probes 130 may be provided in the form of a fiber assembly 125 (see FIG. 14A and FIG. 14B). More details of examples of fiber optic probe 130 in the droplet operations gap of DMF cartridge 110 are shown and described below with reference to FIG. 3 through FIG. 9.

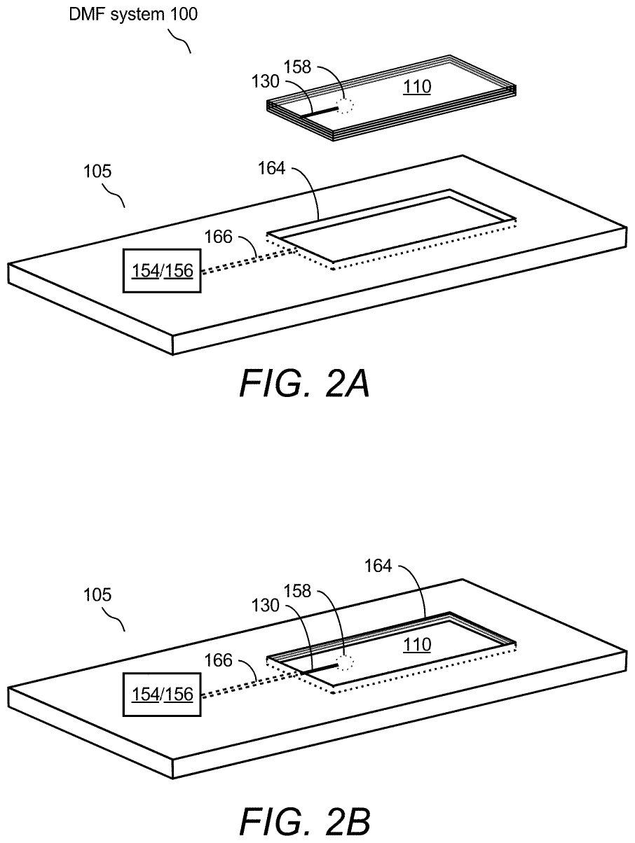

FIG. 2A and FIG. 2B illustrate an embodiment of DMF cartridge 110 in relation to DMF instrument 105 showing an example of an optical fiber interface. In this example, DMF cartridge 110 may be designed to drop-in to a corresponding DMF instrument 105, such as DMF instrument 105. In this example, DMF instrument 105 includes a recessed region 164 for receiving DMF cartridge 110. Namely, DMF cartridge 110 is sized to be fitted into recessed region 164 of DMF instrument 105. In this way, DMF cartridge 110 can be fluidly coupled, optically coupled, and/or electrically coupled to DMF instrument 105. This embodiment is illustrative of a variety of embodiments in which coupling the cartridge to the instrument automatically aligns the fiber optic elements of the cartridge with the fiber optic elements of the instrument. In one embodiment, not shown, alignment may be enhanced by posts or openings in the DMF fiber interface which mate with corresponding posts or openings in the instrument, and a variety of similar approaches that will be apparent to one of skill in the art.

For example, with respect to optical coupling, fiber optic probe 130 of DMF cartridge 110 may include one optical fiber or a bundle of multiple optical fibers. Further, the optical fiber(s) may be multimode or single mode or a combination of the two (with multiple cores and/or claddings layers). DMF cartridge 110 may include one or several interfaces to allow for the coupling of one or more optical fibers to DMF instrument 105. The optical interface(s) may be, for example, fiber optic connectors, fiber optic couplers, and/or free-space optical couplers. For example, FIG. 2A and FIG. 2B show that when DMF cartridge 110 is loaded into recessed region 164 of DMF instrument 105, the outer end of fiber optic probe 130 in DMF cartridge 110 may substantially align with, for example, one or more optical fibers 166 leading from illumination source 154 and/or to optical measurement device 156. The system and cartridge may include alignment elements to ensure that when a DMF cartridge coupled to the instrument an optical connection between the fiber optic elements of the instrument and cartridge are optically coupled. For example, when DMF cartridge 110 is inserted into DMF instrument 105, DMF instrument 105 may automatically perform alignment steps to maximize coupling efficiency between the fibers in DMF cartridge 110 and those in DMF instrument 105. It will be appreciated that a wide variety of mechanisms are possible for electrically and optically coupling the cartridge to the instrument.

FIG. 3, FIG. 4, FIG. 5 show side views of an example of a portion of DMF cartridge 110 of DMF system 100 shown in FIG. 1 and wherein fiber optic probe 130 is introduced into the gap from the top, bottom, and side, respectively. DMF cartridge 110 may include a bottom substrate 112 and a top substrate 114 separated by a droplet operations gap 116. Droplet operations gap 116 may be filled with a filler fluid, such as a gas or a liquid that is sufficiently immiscible with the droplet that it does not substantially interfere with the desired analytical process. In one embodiment, the filler fluid is a low viscosity oil, such as silicone oil or hexadecane. Additionally, an arrangement of droplet operations electrodes 120 (e.g., electrowetting electrodes) may be provided atop bottom substrate 112. DMF cartridge 110 may include any lines or paths of droplet operations electrodes 120.

In one example, bottom substrate 112 may be a material that is substantially transparent to white light (or any color light). For example, bottom substrate 112 may be formed of glass, plastic, or a class of polymers known as thermoplastic elastomers (TPE). In another example, bottom substrate 112 may be a printed circuit board (PCB) that is substantially transparent or one that includes holes or openings that allow light transmission. Like bottom substrate 112, top substrate 114 may be formed of a material that is substantially transparent to white light (or any color light). For example, top substrate 114 may be formed of glass, plastic, or TPE. Further, the inner surface of top substrate 114 may be coated with a conductive layer 118, such as a transparent conductive layer (e.g., indium tin oxide (ITO), poly(3,4-ethylenedioxythiophene) polystyrene sulfonate (PEDOT:PSS)), or other similar transparent or non-transparent (e.g., opaque) conductive coatings. In other embodiments, all areas of DMF cartridge 110 need not include substantially transparent substrates and/or coatings or layers. For example, the substrates and/or coatings or layers may not be transparent, translucent, and/or opaque except in the region of detection. In some cases, such substrates would be unsuitable for free-space optical probes which may require transparent layers. This is an advantage of this present disclosure as the fiber optic probe may be used without transparency, enabling greater flexibility in selecting substrates and fabrication techniques.

The terms “top,” “bottom,” “over,” “under,” “in,” and “on” are used throughout the description with reference to the relative positions of components of the DMF cartridge, such as relative positions of top and bottom substrates of the DMF cartridge. It will be appreciated that the DMF cartridge is functional regardless of its orientation in space.

In DMF cartridge 110, droplet operations gap 116 may be a space for processing any liquids of interest via droplet operations; liquids, such as, but not limited to, liquid reagents, buffer solution, sample fluid, and the like. The gap height may be, for example, a few hundred microns. Droplet operations electrodes 120 may be used for performing droplet operations via electrowetting. “Droplet operation” means any manipulation of a droplet on a digital microfluidics device or cartridge. A droplet operation may, for example, include: loading a droplet into the digital microfluidics device; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a DMF cartridge 110; other droplet operations described herein; and/or any combination of the foregoing. Further, for controlling the temperature of processes occurring in droplet operations gap 116, a temperature control element (not shown), such as a Peltier heat pump, may be used in combination with DMF cartridge 110.

Further, while FIG. 3, FIG. 4, FIG. 5 describe the DMF cartridge 110 as manipulating droplets via electrowetting methods (e.g., using droplet operations electrodes 120), this is exemplary only. In other embodiments, droplets may be manipulated in DMF cartridge 110 via other methods, such as, but not limited to, optical methods, magnetic methods, thermocapillary methods, surface acoustic wave methods, and other electrical methods such as dielectrophoresis, and any combinations thereof.

Referring now to FIG. 3, DMF cartridge 110 of DMF system 100 is shown wherein fiber optic probe 130 is introduced to droplet operations gap 116 from the top (e.g., through top substrate 114 and substantially normal to the plane of DMF cartridge 110). A sample droplet 140 is present in droplet operations gap 116 and atop a certain droplet operations electrode 120. The tip of fiber optic probe 130 may interact directly in/with sample droplet 140, thereby forming an onboard sensing region 158. DMF cartridge 110 may be used to move droplets via droplet operations to and from onboard sensing region 158. The integration of fiber optic probe 130 directly into droplet operations gap 116 and in the path of the sample droplets 140 allows for localized optical interrogation techniques. FIG. 3 shows excitation light 132 being delivered to sample droplet 140 via fiber optic probe 130. Additionally, emission light 134 from sample droplet 140 exits via fiber optic probe 130. Again, fiber optic probe 130 may include one optical fiber or a bundle of multiple optical fibers.

Referring now to FIG. 4, DMF cartridge 110 of DMF system 100 is shown wherein fiber optic probe 130 is introduced into droplet operations gap 116 from the bottom (e.g., through bottom substrate 112 and substantially normal to the plane of DMF cartridge 110).

Referring now to FIG. 5, DMF cartridge 110 of DMF system 100 is shown wherein fiber optic probe 130 is introduced into droplet operations gap 116 from the side (e.g., between bottom substrate 112 and top substrate 114 and substantially parallel to the plane of DMF cartridge 110).

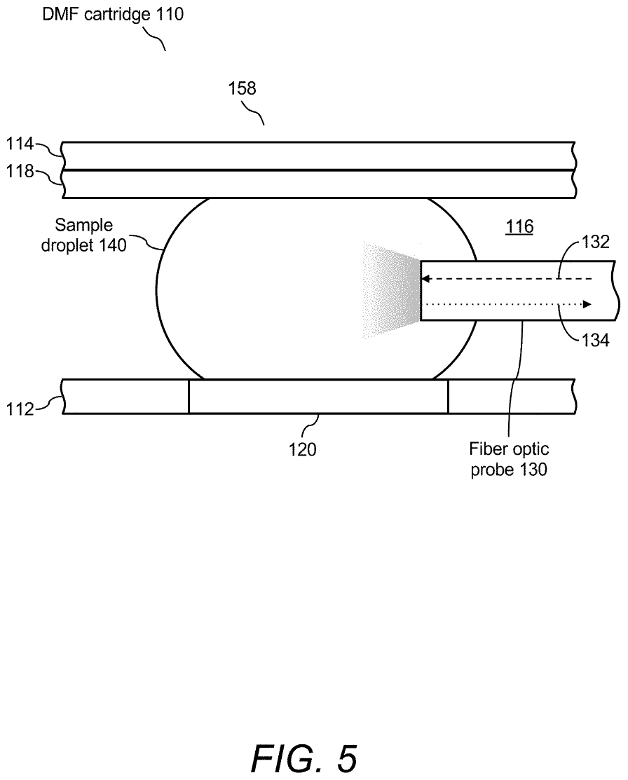

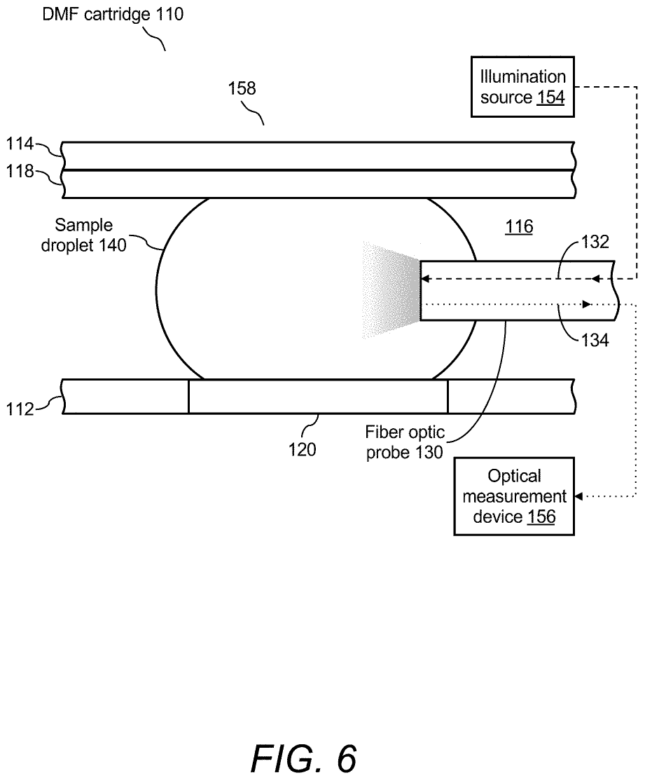

Referring now to FIG. 6 is a side view of an example of DMF cartridge 110 of DMF system 100 wherein fiber optic probe 130 is introduced into droplet operations gap 116 and the optical sensing operations are being performed in reflection mode. While FIG. 6 shows the fiber optic probe 130 configuration shown in FIG. 5, the fiber optic probe 130 configurations shown in FIG. 4 and FIG. 5 are equally applicable with respect to operating in reflection mode.

FIG. 6 shows excitation light 132 from illumination source 154 being delivered to sample droplet 140 via fiber optic probe 130. Additionally, emission light 134 from sample droplet 140 exits via fiber optic probe 130 to optical measurement device 156. This configuration of the DMF system 100 and DMF cartridge 110 provides an optical fiber integrated with a DMF cartridge in a manner that allows for reflective sensing of the liquids' optical properties (e.g., the optical properties of sample droplet 140). Fiber optic probe 130 is part of DMF cartridge 110 that also includes the other components required for digital microfluidics.

Again, illumination source 154 may be one or multiple light sources. The light sources may be monochromatic or polychromatic. These include, but are not limited to, light emitting diodes (LEDs), lasers, incandescent sources, fluorescent sources, or any combinations thereof. The light source may be modulated to have an adjustable intensity very rapidly. The light source may also contain one or more sensors, such as photodetectors, to regulate the brightness. The light source may also contain additional filters to ensure the quality of the incident light. Furthermore, the light source may be a combination of multiple individual light emitting elements. These elements may be active at the same or different wavelengths.

Again, optical measurement device 156 may be one or multiple optical sensors. Optical measurement device 156 may, for example, be a narrowband or a broadband sensor. Optical measurement device 156 converts the optical signal to an electronic signal that may be further processed. Optical measurement device 156 may also use multiple sensors that are sensitive to different parts of the optical spectrum. This could be in the form of a spectrometer or multiple individual sensors that are sensitized to different parts of the electromagnetic spectrum. Optical measurement device 156 may contain additional optical filters as needed to ensure best performance. Optical measurement device 156 may include additional components that allow for the control of illumination source 154 using closed loop control, such as correlators.

In operation, the excitation light 132 from illumination source 154 passes through fiber optic probe 130. Fiber optic probe 130 conducts the incident light to the droplet (e.g., sample droplet 140) being interrogated. Emission light 134 is reflected from the droplet back along the same core of the fiber or a along different core depending on the configuration of the system. Fiber optic probe 130 may have single or multiple cores. These cores may, for example, be single mode or multimode (or any combination of the two). This reflected emission light 134 is coupled to optical measurement device 156.

The configurations of fiber optic probe 130 in DMF cartridge 110 shown in FIG. 3, FIG. 4, FIG. 5 in which the optical sensing operations are being performed in reflection mode enables the use of certain techniques for characterization of the droplet, such as, but not limited to, the following:
(1) Reflectance spectroscopy, such as infrared spectroscopy;
(2) Variable path length spectroscopy;
(3) Absorbance spectroscopy, such as is used for enzyme linked immunosorbent assays (ELISA) readout and polymerase chain reaction (PCR) readout;
(4) Raman spectroscopy;
(5) Photoemission spectroscopy, such as laser induced breakdown spectroscopy and atomic emission spectroscopy;
(6) Fluorescence spectroscopy, such as is used for ELISA readout and PCR readout;
(7) Turbidity measurement;
(8) Time-resolved spectroscopy, such as photon correlation spectroscopy and fluorescence correlation spectroscopy; and
(9) Microscale thermophoresis.

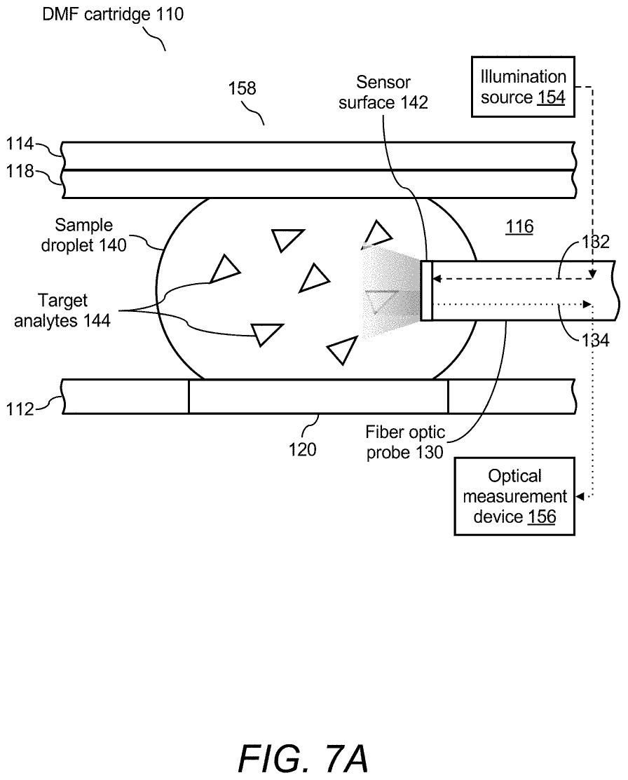

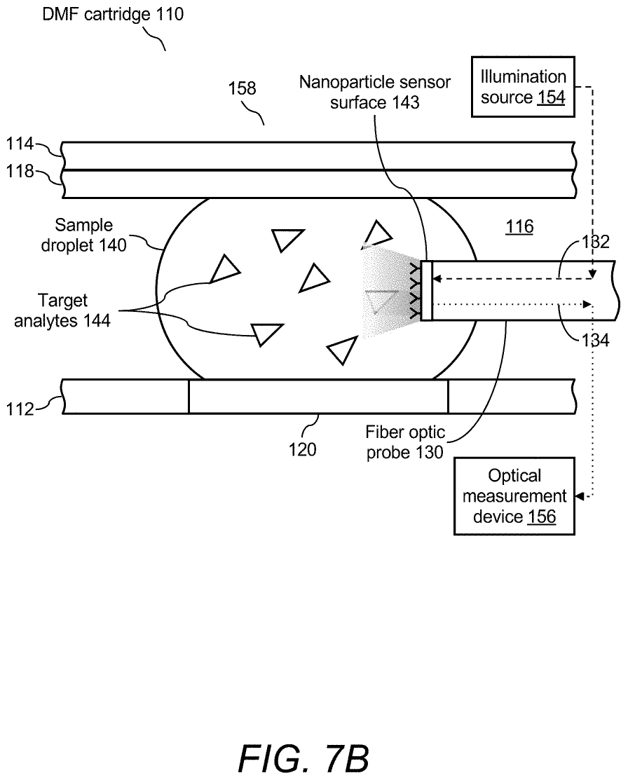

FIG. 7A is a side view of an example of DMF cartridge 110 of DMF system 100 wherein a sensor surface 142 is provided on the tip of fiber optic probe 130 that is in droplet operations gap 116.

A variety of techniques are known to be useful for manufacturing sensors using optical fibers. In one embodiment, the present disclosure makes use of the techniques described in Jeong, Hyeon-Ho & Erdene, Norov & Lee, Seung Ki & Jeong, Dae & Park, Jae-Hyoung. (2011). Fabrication of fiber-optic localized surface plasmon resonance sensor and its application to detect antibody-antigen reaction of interferon-gamma. Optical Engineering. 50. 124405-124405. 10.1117/1.3662418. See also Proll, G., Markovic, G., Steinle, L., & Gauglitz, G. (2009). Reflectometric Interference Spectroscopy. Methods in Molecular Biology: Biosensors and Biodetection, 503, 167-178. doi: 10.1007/978-1-60327-567-5_8. The entire disclosures of Jeong et al. and Proll et al. are incorporated herein by reference.

Sensor surface 142 may be, for example, an optical layer that can convert a property of the droplet or an analyte in the droplet to an optical response. For example, the property converted into an optical response may include presence an analyte, temperature, temperature change, changes in the properties of the analyte or solution, and so on.

In one example, sensor surface 142 may use an interference filter. The interference filter may be deposited onto the fiber using vacuum deposition techniques. The interference filters enable the measurement of binding to the surface using monochromatic intensity or phase measurements or polychromatic spectroscopic measurement.

One example of techniques for fabricating filters useful for the fiber optic probes of the present disclosure is described in Proll G., Markovic G., Steinle L., Gauglitz G. (2009) Reflectometric Interference Spectroscopy. In: Rasooly A., Herold K. E. (eds) Biosensors and Biodetection. Methods in Molecular Biology™, vol 503. Humana Press, the entire disclosure of which is incorporated herein by reference.

In another example, sensor surface 142 may be an interference filter or optical material that may be deposited using wet chemistry. The optical material may include, but is not limited to, metal nanoparticles. Properties, such as surface plasmon resonance or surface-enhanced Raman spectroscopy, may be used to analyze the droplet and/or analytes in the droplet. Other examples of sensor surface 142 may include the deposition of nanostructured surfaces using lithographic techniques. Examples of materials include metallic (e.g. plasmonic nanoparticles), glass (e.g. diffractive elements), plastics (e.g. nanoimprinted diffractive elements). Additionally, sensor surface 142 may be in the form of dielectric microspheres used to focus the excitation light 132 to a smaller region.

Sensor surface 142 may also include a layer that is responsible for binding of analytes from solution to the surface for an enhanced signal. For example, the layer could be deposited on top of the optical sensor surface 142. This layer may also be used without the optical sensor surface 142 as a layer to concentrate the analyte at the tip of the fiber to enhance the optical signal from the analyte. In one example, the layer is a gel matrix that facilitates increased binding sites. In another example, the layer is a porous material to facilitate an increase in the surface concentration of the analyte. Various chemical enhancements will be known to those in the art for increasing binding sites (e.g. hydrophobic sites) or adding specific binding sites (e.g. antibody/antigen or aptamer/analyte). The porous materials may in some cases be organic in nature such as polymer brushes, gels, nanoparticles or other high surface area structures. These organic porous media may me synthetic polymers such as polydimethylsiloxane, divinylbenzene, polyethylene glycol etc. In another embodiment, the porous media may be biologically derived such as cellulose, chitin, collagen etc.

Alternatively, the porous medium could be inorganic in nature such as nanostructured carbon, silica, titania etc. The porous media could be any combination of these materials to harness unique chemical properties of on the analyte to increase their surface concentration.

In another example, sensor surface 142 may be an LSPR sensor layer that is functionalized with one or more capture molecules. The LSPR sensor layer may be comprised of metallic nanostructures and/or multi-layer nanostructures where one of the layers is metallic. Examples of metals typically used for LSPR sensors include nanoparticles of gold, silver, platinum, palladium and copper. In one example, capture molecules are ligands that are immobilized on the surface of LSPR sensor layer. For example, proteins, antibodies, antigens or aptamers as ligands may be attached to a metallic nanoparticle sensor layer, using carboxyl, NTA, or streptavidin surface attachment chemistry. In this example, the ligand is one of the two binding partners, the other binding partner being a target analyte 144 in sample droplet 140.

In another example, the sensor surface 142 may be a pH sensitive optical layer such as fluorescein contained in a gel matrix. The optical properties of these pH sensitive gels is related to the pH of the environment they occur in. As a result, the optical signal readout can change based on the pH of the sample droplet 140.

In another example, the sensor surface 142 may be a thermally sensitive layer such as a thermochromic coating or even an LSPR sensor coated with a passivation layer such as silicon oxide. The optical properties of these sensors are strongly correlated to the temperature of the environment they are in. As a result, the optical signal can change as a function of the temperature of the sample droplet 140

The configuration shown in FIG. 7A wherein a sensor surface 142 is present on the tip of fiber optic probe 130 in DMF cartridge 110 allows certain characterization techniques, such as, but not limited to, the following:
(1) Single color reflectometry;
(2) Reflectometric interference spectroscopy, such as bio-layer interferometry and single color reflectometry (SCORE);
(3) Surface-enhanced Raman spectroscopy;
(4) Surface plasmon resonance;
(5) Diffractive optical measurements;
(6) Solid phase microextraction (coupled with optical readout methods); and
(7) Temperature measurements.

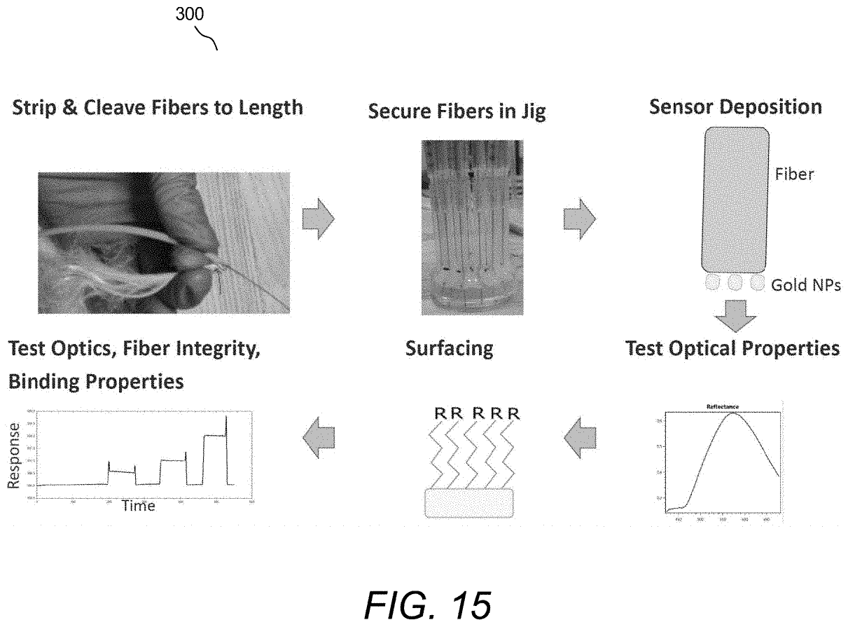

FIG. 7B is a side view of an example of DMF cartridge 110 of DMF system 100 that includes a nanoparticle sensor surface 143 on the tip of the fiber optic probe 130. More details of an example nanoparticle sensor surface 143 are shown below with reference to FIG. 15.

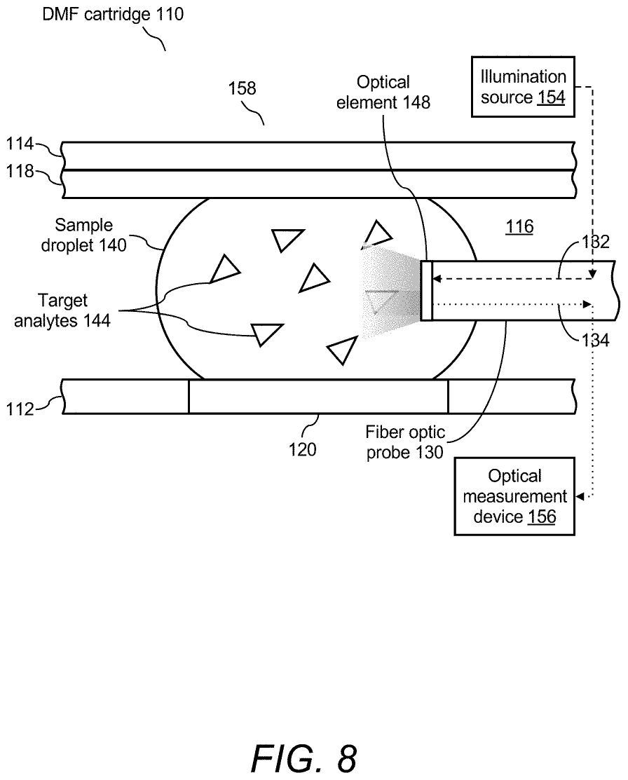

Referring now to FIG. 8 is a side view of an example of DMF cartridge 110 of DMF system 100 wherein one or more optical elements 148 are provided on the tip of fiber optic probe 130 that is in droplet operations gap 116. The one or more optical elements 148 may be provided alone or in addition to sensor surface 142 shown in FIG. 7. The one or more optical elements 148 may be used to more effectively couple the light into and/or out of sample droplet 140 and/or sensor surface 142. The one or more optical elements 148 may include, but are not limited to, the following:
(1) Tapered optical fiber, such as a conical taper;
(2) U-shaped optical fiber;
(3) Lenses, such as hemispherical lenses, ball lenses, GRIN lenses, and aspheric lenses;
(4) Prisms, such as ATR prisms and Kretschmann prisms;
(5) Angled polish, such as side polished and wedge polished;
(6) Diffusing elements; and
(7) Reflective cavity, such as Fabry-Perot etalon and transflection cavity.

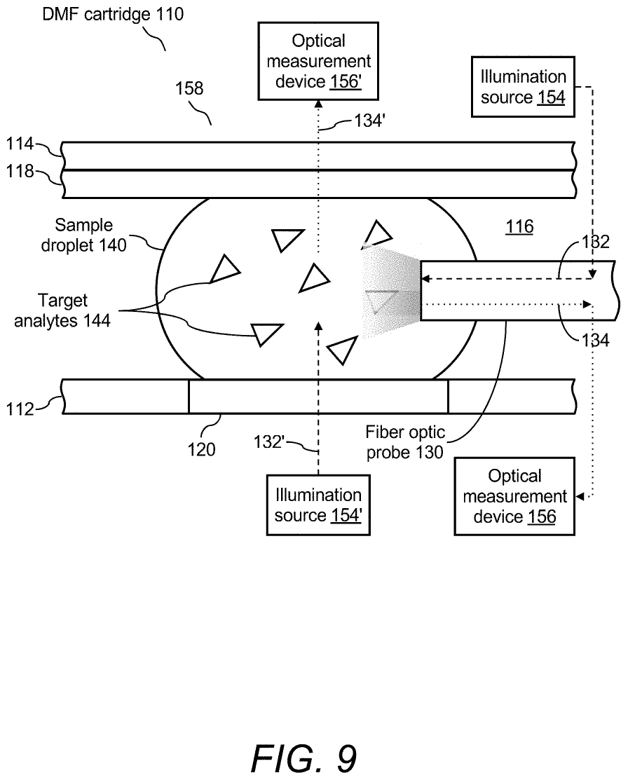

FIG. 9 is a side view of an example of DMF cartridge 110 of DMF system 100 that is configured substantially the same as that shown in FIG. 6 except for the addition of a secondary illumination source 154′ and a secondary optical measurement device 156′. In this example, illumination source 154′ and/or optical measurement device 156′ may be used independently or in conjunction with fiber optic probe 130 coupled to illumination source 154 and optical measurement device 156.

Like optical measurement device 156, optical measurement device 156′ may be in the form of a light measuring device, such as a photodetector, a camera, a spectrometer or a hyperspectral imager. Optical measurement device 156′ may take time resolved measurements. Further, input from optical measurement device 156′ may be used to control illumination source 154 and/or illumination source 154′.

The presence of the secondary illumination source 154′ and the secondary optical measurement device 156′ allow for a variety of operating modes. In one example, using the primary illumination source 154 and the primary optical measurement device 156, optical sensing operations may be performed in reflection mode entirely through fiber optic probe 130. That is, both illuminate and collect using fiber optic probe 130. In another example, using the primary illumination source 154 and the secondary optical measurement device 156′, optical sensing operations may be performed in transmission mode. That is, illuminate using fiber optic probe 130 and the primary illumination source 154 but collect using the external secondary optical measurement device 156′. In yet another example, using the secondary illumination source 154′ and the primary optical measurement device 156, optical sensing operations again may be performed in transmission mode. That is, illuminate using the external secondary illumination source 154′ but collect using fiber optic probe 130 and the primary optical measurement device 156.

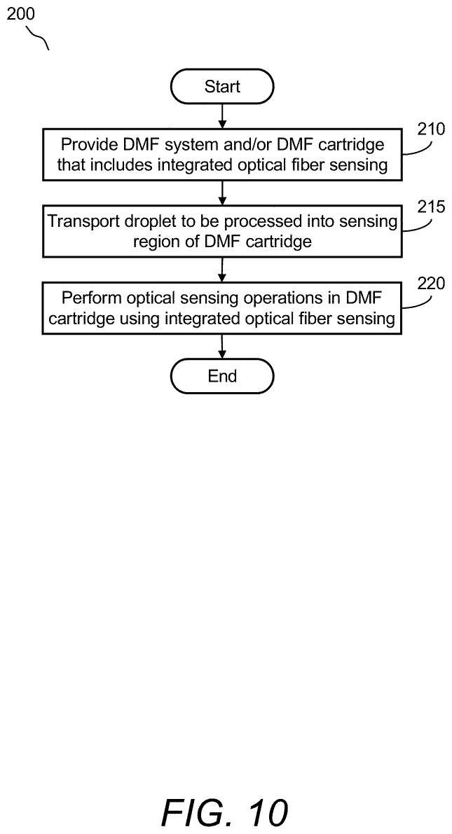

Referring now to FIG. 10 is a flow diagram of an example of a method 200 of using the DMF system 100 and/or DMF cartridge 110 that includes integrated optical fiber sensing. Method 200 may include, but is not limited to, the following steps.

At a step 210, a DMF system and/or DMF cartridge that includes integrated optical fiber sensing is provided. For example, the DMF system 100 and/or DMF cartridge 110 that includes fiber optic probe 130 integrated in the droplet operations gap 116 of DMF cartridge 110 is provided, as shown, for example, in FIG. 1 through FIG. 9.

At a step 215, a droplet to be processed is transported into the sensing region of the DMF cartridge. For example and referring now to FIG. 3 through FIG. 9, a sample droplet 140 to be processed may be transported using droplet operations into onboard sensing region 158 of DMF cartridge 110, wherein onboard sensing region 158 is at the tip of fiber optic probe 130.

At a step 220, optical sensing operations are performed in the DMF cartridge using integrated optical fiber sensing. For example and referring now to FIG. 6 through FIG. 9, optical sensing operations are performed in DMF cartridge 110 using fiber optic probe 130 integrated in the droplet operations gap 116 of DMF cartridge 110, illumination source 154 and optical measurement device 156. In operation, the excitation light 132 from illumination source 154 passes through fiber optic probe 130 and is directed toward and into the sample droplet 140 being interrogated. Emission light 134 is then reflected from the sample droplet 140 back along fiber optic probe 130 to optical measurement device 156, which captures the optical reading for processing. In one example, the optical sensing operations in DMF cartridge 110 may occur in reflection mode, as shown in FIG. 6, FIG. 7, and FIG. 8. In another example, the optical sensing operations in DMF cartridge 110 may occur in reflection mode, transmission mode, or both reflection mode and transmission mode, as shown and described in FIG. 9.

In certain embodiments, it may be useful to oscillate the droplet while it is in contact with the sensor. For example, in one embodiment, first reaction electrode and the second reaction electrode are alternately activated to induce oscillation of the droplet between the first reaction electrode and the second reaction electrode to induce the movement of the droplet relative to the SPR sensor surface. In one embodiment, the oscillation of the droplet between the first reaction electrode and the second reaction electrode is linear. In one embodiment, the oscillation ranges from about 0.5 to about 15 Hz. In another embodiment, the oscillation ranges from about 4 to about 10 Hz.

In another embodiment, the SPR sensor surface is disposed between three or more reaction electrodes, and the three or more reaction electrodes are alternately activated to induce oscillation of the droplet between the three or more reaction electrodes to induce the movement of the droplet relative to the SPR sensor surface. In another embodiment, the oscillation of the droplet between the three or more reaction electrodes is circular.

In one embodiment, a set of electrodes is arranged relative to the sensing tip of the sensor such that oscillation by moving the droplet from one electrode to the next electrode in the set retains the droplet in contact with the sensing tip of the sensor.

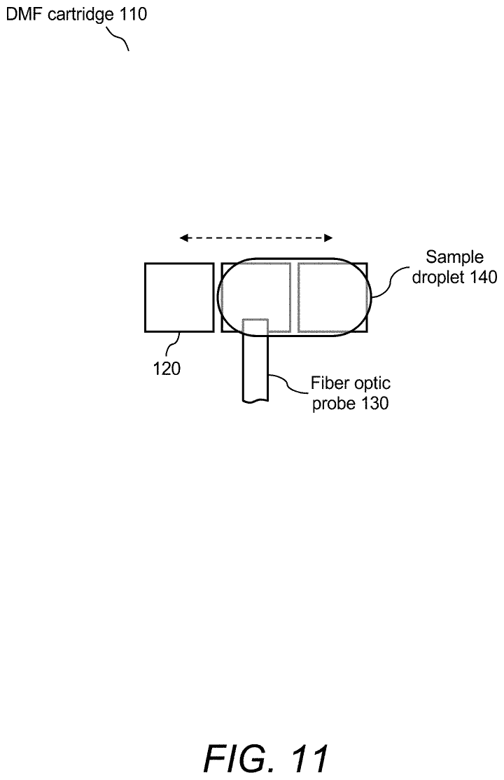

FIG. 11 shows an example of 2X sample droplet 140 oscillating back and forth atop droplet operations electrodes 120 and in contact with fiber optic probe 130. In FIG. 11, the droplet is generally moving along the path of droplet operations electrodes 120 in a direction which is at a right angle to a line (not shown) along the length of the fiber optic probe 130. It will be appreciated that other kinds of movement will be possible within the scope of the present disclosure, e.g., a direction congruent with a line (not shown) along the length of the fiber optic probe 130, a variety of directions. Also, oscillation may include elongating and shortening the droplet. Further, while the image shows 2X elongation, it will be appreciated that elongation may be 3X, 4X, or even longer (X=the number of activated electrodes used to elongate the droplet).

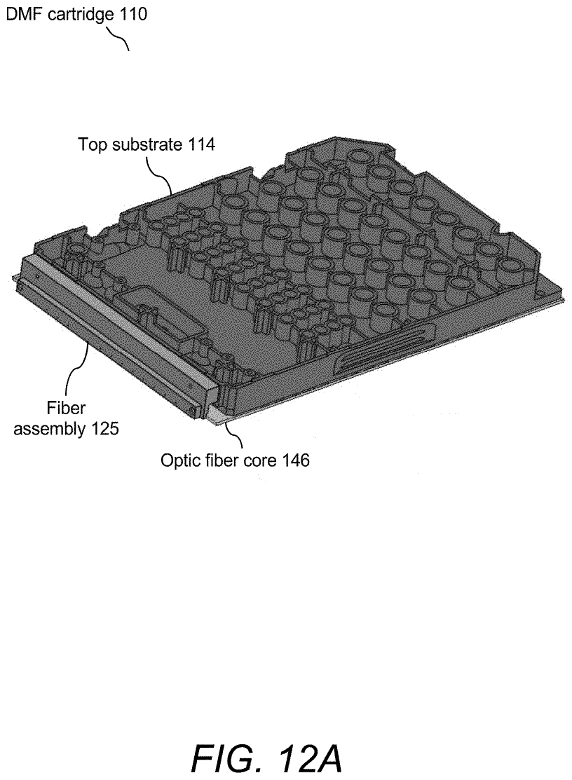

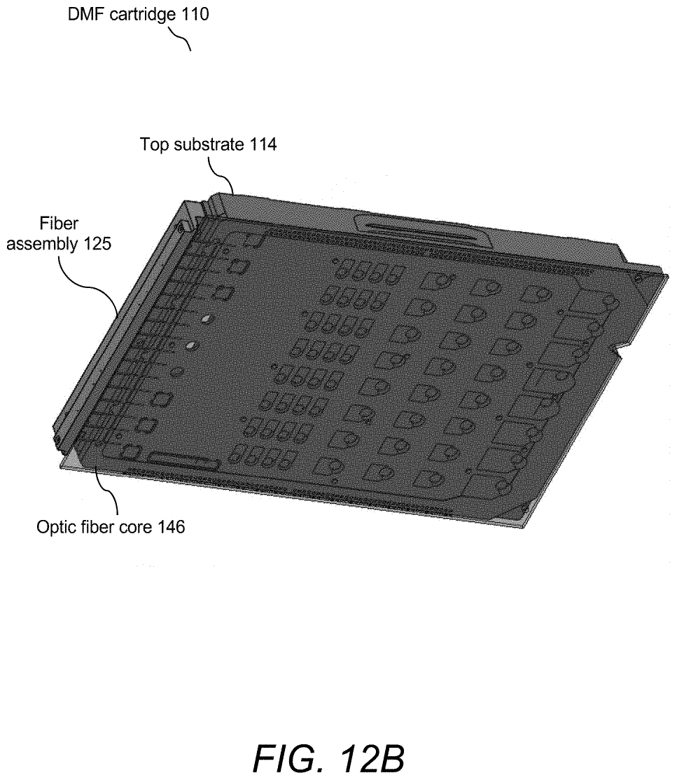

FIG. 12A and FIG. 12B illustrate a top perspective view and a bottom perspective view, respectively, of an example instantiation of DMF cartridge 110 that includes integrated fiber sensing. For example, FIG. 12A and FIG. 12B show an example of top substrate 114, fiber assembly 125, and optic fiber core 146 of DMF cartridge 110.

Fiber assembly 125 may be configured such that a set of fibers extend from a first edge of the assembly and extend from a second edge of the assembly. When fiber assembly 125 is assembled with DMF cartridge 110 the droplet operations gap 116 between the top substrate 114 and bottom substrate 112 is sealed, and the fiber optic probes extend into the gap. Ideally the fibers extend into sufficient proximity with one or more electrowetting electrodes 120 that a droplet interacting with one or more electrowetting electrodes 120 will contact the end of the fiber optic probe. For assays requiring oscillation of droplets, the set of electrodes may be arranged relative to the sensing tip of the probe such that oscillation by moving the droplet from one electrode to the next electrode in the set retains the droplet in contact with the sensing tip of the sensor.

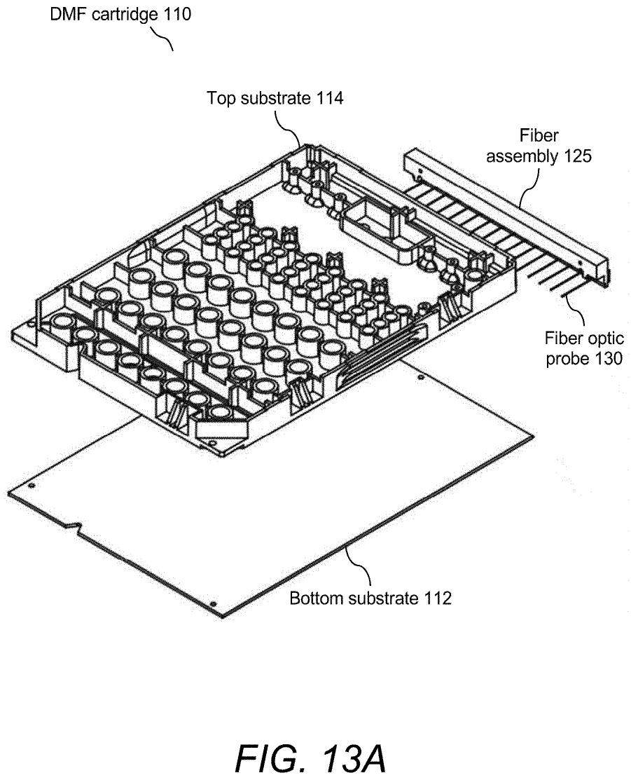

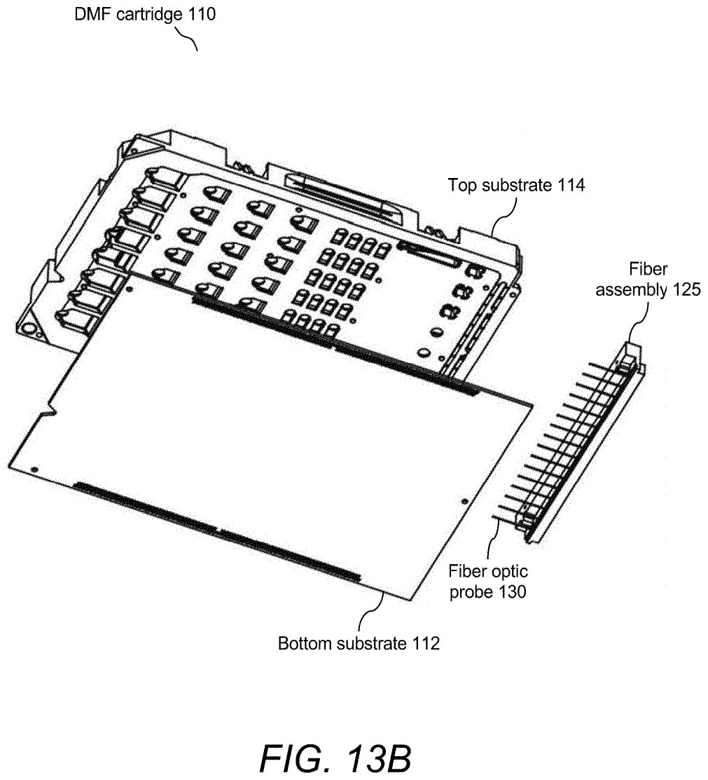

FIG. 13A and FIG. 13B illustrate a top exploded view and a bottom exploded view, respectively, of an example of DMF cartridge 110. Top substrate 114 contains openings for depositing buffers, reagents and samples into DMF cartridge 110. The openings are spaced apart to enable the use of multichannel pipettors for the introduction of reagents, buffers and samples into DMF cartridge 110. The footprint of DMF cartridge 110 may be optimized for use with equipment used of well plates. Bottom substrate 112 is a printed circuit board with electrodes patterned in it to control the actuation of the droplets. Bottom substrate 112 has a hydrophobic coating to optimize the electrowetting force for DMF. Top substrate 114 is made from an injection moulded polymer, such as polycarbonate, acrylic, cyclic olefin copolymers etc. Top substrate 114 has a conductive coating applied such as indium tin oxide to enhance its conductivity. Top substrate 114 is sealed to bottom substrate 112 using elastomeric adhesives such as silicone. The fibers are introduced into the cartridge via v-grooves in top substrate 114 that help align the fibers. The elastomeric adhesive mentioned above also seals around the fibers as they enter into the gap between top substrate 114 and bottom substrate 112. Fiber assembly 125 is secured onto top substrate 114 either using adhesives, fasteners, ultrasonic welding, heat staking or other methods of fastening

FIG. 14A and FIG. 14B illustrate various views of an example of fiber assembly 125 of DMF cartridge 110. FIG. 14A shows an exploded view and a cutaway view of fiber assembly 125. FIG. 14B shows a perspective view of fiber assembly 125. Fiber assembly 125 may include two primary components; multiple optical fibers 180 (i.e., for forming fiber optic probes 130, and a fiber holder 182. Fiber holder 182 also includes two alignment holes 184, one at each end. To fabricate fiber assembly 125, optical fibers 180 are cut to length and glued into fiber holder 182. Optical fibers 180 extend about 20 mm out one face of fiber holder 182 (the sensor end) and about 5 mm out the other side (an instrument mating face 186). The excess fiber on the instrument side may be cut with a ruby scribe to be flush with instrument mating face 186 and then polished down. The other end (the sensor end) if optical fibers 180 is cleaved to length. The sensor end then undergoes further processing to form fiber optic probes 130. For example, processing may involve coating nanoparticles on the tip of the sensor end.

In summary and referring now again to FIG. 1 through FIG. 14B, the DMF system 100, DMF cartridge 110, and/or method 200 uses optical fibers (e.g., fiber optic probe 130) integrated directly into a disposable cartridge (e.g., DMF cartridge 110) to allow for localized optical interrogation techniques. Compared with conventional methods, the integration of optical fiber sensors with digital microfluidics in DMF system 100 allows for localized optical interrogation techniques, provides a low-cost method of achieving high optical performance (e.g., reduced component count for lower cost), limits light loss in the system for improved signal, and limits collection of stray light for lower noise.

### EXAMPLES

### Example 1

Referring now to FIG. 15 is an example of a process 300 of making a sensor fiber. Process 300 may include, but is not limited to, the steps of (1) strip and cleave fibers to length, (2) secure fibers in jig, (3) deposit sensors on tip of fibers, and (4) test optical properties, fiber integrity, and binding properties. This is one example of how to provide make a fiber optic probe for insertion into the droplet operations gap of a DMF cartridge.

### Example 2

This experiment was to determine the affinity of protein-A and IgG, a common protein-protein interaction, using an optical fiber-based surface plasmon resonance detection method within a DMF device. The experiment demonstrated both the autonomous processing and low-volume advantages of a DMF device as well as the low-background, limited components and alignment advantages of the optical-fiber detection.

Materials

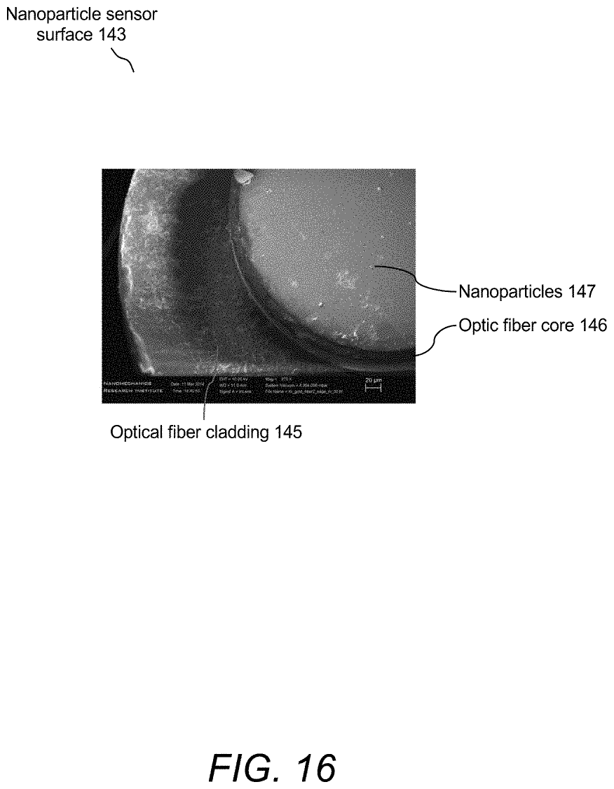

125 μm diameter optic fibers were cleaved and processed to deposit gold nanoparticles on the fiber tip and then surface those nanoparticles with carboxyl groups. Briefly, the optical fiber was cleaned thoroughly and oxylated. Example methods of oxylation include exposure to highly basic solutions such as sodium hydroxide, ozone exposure or oxygen plasma exposure. Next, a self-assembled interface layer is deposited to bind the gold to the sensor. In this example we used mercapto silane compounds such as (3-Mercaptopropyl)trimethoxysilane. Finally, the optical fiber was immersed into a solution containing gold nanoparticles of the desired size and shape. In this case, we used 10 nm diameter gold nanoparticles. After gold deposition, thiol compounds were used to present functional groups for future chemistry. A variety of compounds can be used, e.g., compounds with a thiol group on one side and a hydroxy group on the other will present a carboxyl surface for future chemistry. We used 3-Mercapto-1-propanol. For example, FIG. 16 shows more details of an example of nanoparticle sensor surface 143 (see FIG. 7B). FIG. 16 shows an electron micrograph of a tip of the resulting fiber optic probe showing the optical fiber cladding 145, the optical fiber core 146 and the nanoparticle coating 147.

After surfacing, the sensors were inserted into the gap of a DMF cartridge such that the tips aligned to the edge of an electrode. Each channel of data collected refers to a different fiber optic sensor.

The DMF cartridge was loaded with a 2 centistokes polydimethylsiloxane to provide an oil-environment to the DMF device. The wells of the DMF device were filled with 100 μL of phosphate buffered saline (PBS) at pH 7.4, 35 μL of ethanolamine, 35 μL of glycine+hydrochloric acid (HCl) at pH 1.5, 35 μL of protein A (the ligand used). Also loaded into the cartridge was 8 μL of 10% glycerol in PBS and 16% glycerol in PBS for fiber sensitivity calibration, 8 μL of 1-ethyl-3-(-3-dimethylaminopropyl) carbodiimide hydrochloride (EDC) and 8 μL of N-hydroxysuccinimide (NHS) for activating the carboxyl surface of the sensor and 8 μL of 900 nM IgG for analysis. However, only 2 μL is required for this protocol. All reagents included 0.1% Tween-20 surfactant to stabilize the oil-aqueous interface.

The droplet was oscillated while in contact with the probe as illustrated in FIG. 11. The electrowetting electrodes were used to elongate the droplet to a 2X droplet and shuttle it back and forth over three electrodes at 10 Hz for 10 minutes while maintaining contact with the probe.

Methods

The entire experiment was executed automatically by the DMF cartridge. First, the sensitivity of the sensors was calibrated with the glycerol. This procedure was to:
(1) Introduce 700 nL PBS for baseline;
(2) Measure the refractive index shift of 700 nL 10% glycerol;
(3) Rinse the sensor with 700 nL PBS;
(4) Measure the refractive index shift of 700 nL of 16% glycerol; and
(5) Rinse the sensor with 700 nL PBS.

The above assesses how much signal shift a known refractive index provides. Next, the cartridge automatically measures the binding kinetics of the system. This involved the following steps. Note that many steps were executed in parallel with others for time optimization:
(1) Condition sensors with 700 nL PBS;
(2) Clean the sensors with 700 nL Glycine-HCl;
(3) Rinse fibers with 700 nL PBS;
(4) Mix 350 nL 1-Ethyl-3-(3-dimethylaminopropyl)carbodiimide with 350 nL unit N-Hydroxysuccinimideon each channel and activate the sensor's carboxyl surface with the mixture;
(5) Rinse fibers with 700 nL PBS;
(6) Introduce 700 nL of the protein A solution to each sensor. This will immobilize the protein A on the sensor surface where it will function as a ligand for a protein A antibody;
(7) Rinse the fibers with 700 nL PBS;
(8) Create the IgG sample by:
   (a) Taking 350 nL of 900 nM IgG and diluting it with 700 nL of PBS. Mix the combination thoroughly and divide into 700 nL of the mixture on one side to create the 300 nM sample;
   (b) Take the remaining 350 nL from 8a and dilute it with 700 nL of PBS Similarly, mix the combination and divide into 700 nL to set aside for the 100 nM sample and 350 nL drop for further dilution; and
   (c) Repeat 8b to create the 33 nM, 11 nM and 3.67 nM samples. The remaining 350 nL drop is pushed to waste.
(9) Introduce the 700 nL IgG samples to the various sensors. Measure the association of the IgG to the protein A in real-time; and
(10) Rinse the fibers with 700 nL of PBS. Measure the dissociation of the IgG from the protein A in real-time.

Results and Analysis

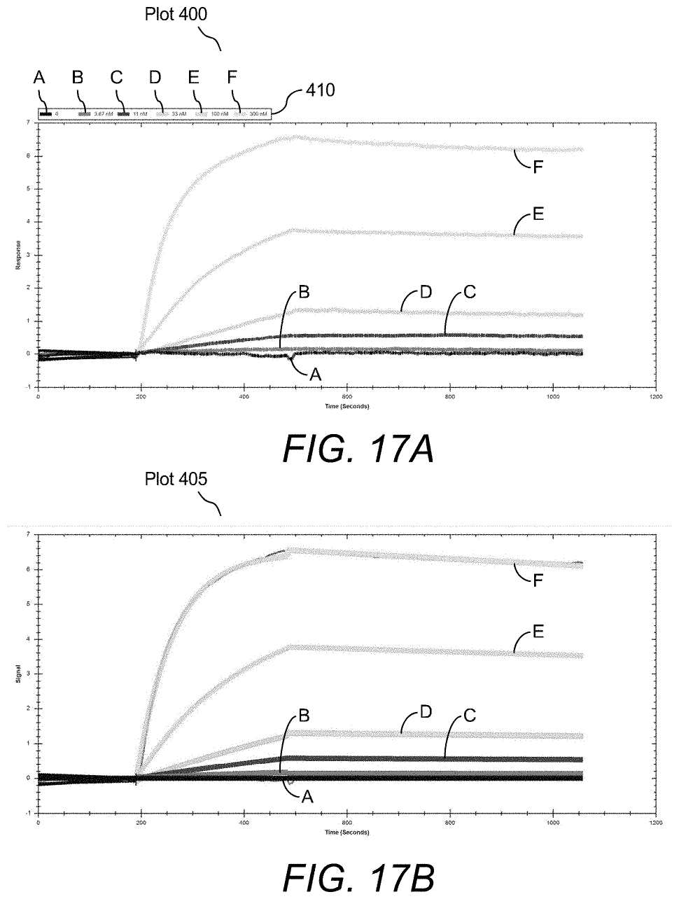

The steps above were used to generate response curves for each IgG sample. These curves were adjusted first for the sensitivity to refractive index shift obtained from the glycerol correction, then for the amount of protein A immobilized on the sensor measured during the experiment. The results are indicated in a plot 400 shown in FIG. 17A. Plot 400 shows response curves A, B, C, D, E, and F. A legend 410 indicates response curve A corresponds to a 0 nM affinity, response curve B corresponds to a 3.67 nM affinity, response curve C corresponds to an 11 nM affinity, response curve D corresponds to a 33 nM affinity, response curve E corresponds to a 100 nM affinity, and response curve F corresponds to a 3000 nM affinity.

A 1:1 kinetics model was applied to this data and fit. As indicated in a plot 405 shown in FIG. 17B, this results in a very close correlation and a measured affinity of 2 nM which is consistent with measurements of the same sample on other instruments. However, unlike other instruments this entire experiment including sample preparation was executed autonomously on a disposable fluidic cartridge with no risk of cross-contamination with previous or subsequent samples. Furthermore, the experiment consumed only 350 nL of the analyte solution, a dramatic decrease in volume compared to other systems.

The present invention may be implemented using hardware, software, or a combination thereof and may be implemented in one or more computer systems or other processing systems. In one aspect, the invention is directed toward one or more computer systems capable of carrying out the functionality described herein.

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present disclosure.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

The terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The terms “comprise,” “comprises,” “comprising,” “include,” “includes,” and “including,” are intended to be non-limiting, such that ensuing recitation of items in a list is not to the exclusion of other like items that may be substituted or added to the listed items.

All publications mentioned in the above specification are herein incorporated by reference.

Various modifications and variations of the disclosed methods, compositions and uses of the invention will be apparent to the skilled person upon review of this disclosure and claims without departing from the scope and spirit of the invention. Although the invention has been disclosed herein in connection with specific preferred embodiments, it should be understood that the invention as claimed should not be unduly limited to such specific embodiments.

1. (canceled)

2. A cartridge for use with an instrument, the cartridge comprising:
a digital microfluidics substrate comprising a plurality of electrowetting electrodes operative to perform droplet operations on a liquid droplet in a droplet operations gap;
a top plate separated from the digital microfluidics substrate to form a droplet operations gap and comprising openings for flowing liquids into the droplet operations gap; and
a fiber assembly comprising a fiber optic probe projecting into the droplet operations gap and having a sensing end situated in proximity with one or more of the electrowetting electrodes.

3. The cartridge of claim 2, wherein a fiber optic probe projecting into the droplet operations gap is situated in proximity with a set of two or more of the electrowetting electrodes such that a droplet situated atop any electrode of the set of two or more electrodes will contact the fiber optic probe.

4. The cartridge of claim 2, wherein the probe comprises a ligand.

5. The cartridge of claim 2, further comprising:
a droplet controllable by the electrowetting electrodes to contact the fiber optic probe.

6. (canceled)

7. The cartridge of claim 2, wherein the top plate comprises two or more grooves or openings each aligning a fiber optic probe from the fiber assembly.

8. The cartridge of claim 2, wherein the sensing end comprises a nanoparticle sensor surface.

9. A method of conducting an assay, comprising:
providing a fiber optic probe comprising a ligand;
providing a droplet having a volume which is less than about 1000 nL and comprising an analyte potentially having an affinity for the ligand; and
contacting the droplet with the end of the probe and oscillating the droplet without removing the droplet from contact with the probe.

10. The method of claim 9, wherein the oscillation ranges from about 0.5 to about 15 Hz.

11. (canceled)

12. The method of claim 9, wherein the droplet has a volume which is less than about 900 nL.

13.-17. (canceled)

18. The method of claim 9, further comprising:
measuring a signal from the droplet via the fiber optic probe and using a computer to calculate a response curve from the signal.

19. The method of claim 9, wherein the fiber optic probe comprises multiple ligands and the droplet comprises multiple analytes.

20. The method of claim 9, wherein the providing operation further comprises providing multiple fiber optic probes and multiple droplets and contacting each of the multiple droplets with a corresponding fiber optic probe and oscillating each of the multiple droplets in contact with the corresponding fiber optic probe.

21. The method of claim 9, wherein the oscillation is mediated by electrowetting electrodes.

22.-23. (canceled)

24. The method of claim 9, wherein the oscillation is mediated by electrowetting electrodes in a droplet operations gap of a digital microfluidics (DMF) cartridge.

25. The method of claim 9, wherein the oscillation is substantially at a right angle to a line extending along a length of the fiber optic probe.

26. The method of claim 9, wherein the oscillation is substantially in line with a line extending along a length of the fiber optic probe.

27. The method of claim 9, wherein the oscillation is multidirectional.

28. (canceled)

29. The method of claim 9, wherein the oscillation is conducted using an elongated droplet.

30.-31. (canceled)

32. The method of claim 9, wherein the assay is selected from the following: molecular library screening assays, binding kinetics assays, affinity determination assays, binding site mapping assays, thermodynamics studies, sandwich assays, competition analysis assays, specificity determination assays, and characterizations of antibody binding, and combinations thereof.

33. A DMF system, the system comprising:
a cartridge, comprising:
digital microfluidics comprising a plurality of electrowetting electrodes operative to perform droplet operations on a liquid droplet in a droplet operations gap, and
a fiber optic probe projecting into the droplet operations gap into proximity with a set of two or more of the electrowetting electrodes such that a droplet situated atop any electrode of the set of two or more electrodes will contact the fiber optic probe;
a controller operationally coupled to the electrowetting electrodes and the fiber optic probe;
an illumination source arranged to illuminate one or more droplets in the droplet operations gap; and
an optical measurement device optically coupled to the fiber optic probe and arranged to receive signal from the fiber optic probe.

34.-47. (canceled)
